(19) United States
(12) Patent Application Publication
(10) Pub. No.: US 2004/0254385 A1
(43) Pub. Date: Dec. 16, 2004

(54) Process for the preparation of citalopram hydrobromide

(76) Inventors: Swargam Sathyanarayana, Gurgaon (IN); Yatendra Kumar, Gurgaon (IN); Tarum Kant Sharma, Muzzaffarpur (IN); Sujay Biswas, West Bengal (IN); Bakthavthsalan Vijayraghavan, Gurgaon (IN)

Correspondence Address: RANBAXY INC., 600 COLLEGE ROAD EAST, SUITE 2100, PRINCETON, NJ, 08540, US

(21) Appl. No.: 10/484,296

(22) PCT Filed:
(86) PCT No.: PCT/IB02/02728

(30) Foreign Application Priority Data
Jul. 19, 2001 (IN) 779/DE;/2001

(51) Int. Cl. C07D 307/78

(52) U.S. Cl. 549/467

(57) ABSTRACT

The present invention relates to an industrially advantageous process for the preparation of pure citalopram hydrobromide.

### FIELD OF THE INVENTION

The present invention relates to an industrially advantageous process for the preparation of pure citalopram hydrobromide.

### BACKGROUND OF THE INVENTION

Citalopram hydrobromide of formula I,

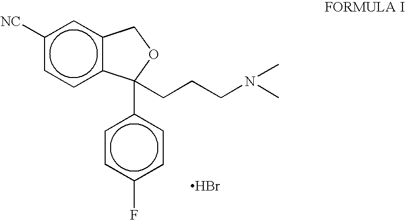

is a well known antidepressant drug and is chemically known as 1-[3-(dimethylamino)propyl]-1-(4-fluorophenyl)-phthalancarbonitrile hydrobromide salt. Citalopram was disclosed for the first time in U.S. Pat. No. 4,136,193 and is known to be a selective centrally acting serotonin reuptake inhibitor. Citalopram has further been shown to be effective in the treatment of dementia and cerebrovascular disorders as disclosed in European Patent No. 474580.

Several processes are known for the preparation of citalopram. However, these processes are not satisfactory as most of these require raw materials which are either expensive or have limited commercial availability, while others involve a large number of synthetic steps. U.S. Pat. No. 4,136,193 outlines a process for the preparation of citalopram which involves ring closure of the compound of Formula Ill

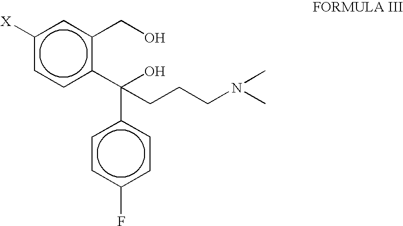

wherein X is a halogen atom,

in the presence of a dehydrating agent and subsequent exchange of 5-halo group with a cyano group using cuprous cyanide.

Variants of this method are disclosed in PCT applications, WO 00/13648 and WO 00/11926 wherein the cyanide exchange is achieved with a cyanide source in the presence of a palladium or nickel catalysts. We have recently reported an improved process for the preparation of citalopram in an Indian patent application (No. 264/Del/2001) which process involves cyanide exchange in the presence of an organic base. The base is believed to form a complex with the cyanide source which facilitates the exchange of halogen with nitrile thus providing an efficient process.

However, we have observed that citalopram obtained from any of the above cited processes contains an impurity which has been now characterized as descyano citalopram of Formula IV,

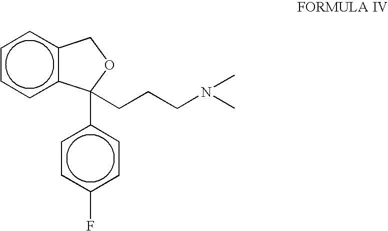

The descyano citalopram impurity is formed as a result of the side reaction of residual magnesium at the 5-position of the 5-halophthalide during the two successive Grignard reactions involved in the preparation of the compound of Formula III.

Another impurity generated during the cyanide exchange process is the 5-carbamoylphthalane of Formula II

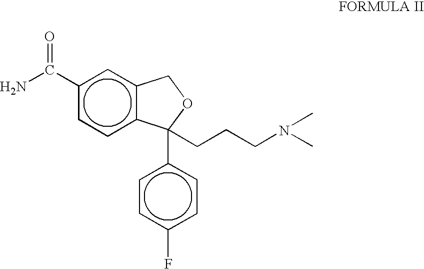

Also, the starting 5-halophthalide does not react completely during the cyanation step and is thus obtained as an impurity in the product.

The pharmaceutical compounds are required in highly pure form because of the fear of unknown and potentially harmful effects of impurities. For purposes of patients' safety, it is highly desirable to limit the amount of impurities present in any medicament administered to a patient. This is achieved by either devising a process to yield a pure product or by using additional purification techniques like chromatography, crystallization etc.

The citalopram base is obtained as an oil and our attempts at removing the descyano citalopram impurity and other impurities formed during the cyanide exchange process by various purification techniques e.g. crystallization, column chromatography proved to be unsuccessful. The removal of impurities by vacuum distillation of the high boiling citalopram is unsuitable to operate on an industrial scale and is uneconomical.

Therefore, it is an objective of the present invention to solve the problems associated with the prior art and devise a simple and efficient process for making citalopram free of the descyano citalopram impurity and other related impurities.

### SUMMARY OF THE INVENTION

Accordingly, the present invention provides a process for the preparation of citalopram hydrobromide of Formula I,

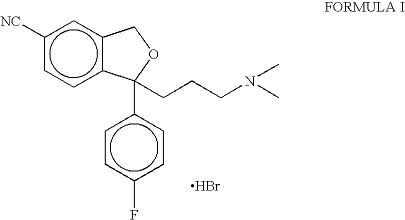

comprising:

i. converting crude citalopram to the corresponding 5-carbamoylphthalane of Formula II, and

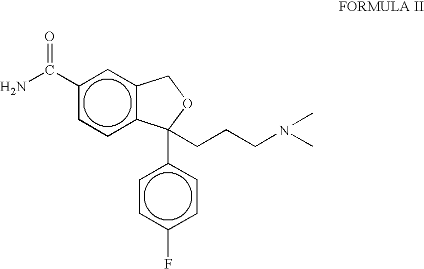

ii. reacting it with a dehydrating agent to obtain citalopram which is converted into its hydrobromide salt.

The conversion of crude citalopram to its corresponding amide of Formula is simple and efficient. The process comprises reacting crude citalopram with a base in the presence of an alcohol, a glycol, a glycol ether, or a mixture thereof.

The base is preferably selected from alkali metal hydroxides such as sodium hydroxide, potassium hydroxide, or lithium hydroxide.

Alcohols may be selected from straight or branched chain C₁-C₈ alkyl alcohols such as ethanol, isopropanol, tert-butanol and neo-pentanol. The reaction may also be performed in a glycol such as monoethylene glycol or in a glycol ether such as diglyme.

The reaction may be performed at room temperature or at higher temperature, preferably at 40° C. to 100° C.

The base may be used in catalytic amounts or in excess. The base used is preferably 0.2 to 2.5 molar equivalents with respect to the starting citalopram.

The 5-carbamoylphthalane of Formula II is isolated by suitable aqueous work-up. The reaction mixture is poured into water, extracted with a solvent such as ethyl acetate or dichloromethane and the solvent is evaporated to obtain the product. However, the crystalline 5-carbamoylphthalane of Formula II is obtained by trituration of the residue with toluene followed by the addition of hexane.

The 5-carbamoylphthalane compound of Formula II may also be obtained from impure citalopram by any method known in the art, such as hydrolysis of impure citalopram to its corresponding carboxylic acid followed by its esterification and subsequent amidation with ammonia as reported in Eur. J. Med. Chem. Ther. 12(3), 289-295 (1977).

The cyano group of the impure citalopram may also be directly converted to the amide group of the 5-carbamoyphthalane of Formula II by conventional methods known in synthetic organic chemistry e.g., Comprehensive Organic Transformation; VCH; New York, p.993 (1989).

The dehydration of the 5-carbamoylphthalane of Formula II to citalopram may be achieved by reaction with any of the dehydrating agents such as thionyl chloride, phosphoryl chloride, phosphorous pentachloride, polyphosphoric acid, phosphorous pentoxide or a Vilsmeier reagent. Thionyl chloride is preferred.

The dehydration may be performed without a solvent or in an inert solvent. Suitable solvents include hydrocarbons such as toluene and chlorinated hydrocarbons such as dichloromethane.

The dehydration may be performed at higher temperatures, preferably at 50-100° C.

The hydrobromide salt of citalopram may be prepared by methods known in the art. The base is reacted with either a calculated amount of acid in a water miscible solvent such as ethanol or acetone and the salt isolated after concentration and cooling, or with an excess of the acid in a water immiscible solvent such as ether, dichloromethane or toluene with the salt separating out spontaneously.

In the meaning of the present invention, “pure citalopram hydrobromide” includes citalopram hydrobromide having a purity of 99.0% or more by HPLC. Also, the citalopram hydrobromide obtained by the process of the present invention contains less than 0.2% of the descyano citalopram impurity.

### DETAILED DESCRIPTION OF THE INVENTION

The invention is further illustrated by the following examples which should not be construed to be limiting the scope of the present invention.

### EXAMPLE 1

### Preparaton of 1-[3-Dimethylamino)propyl]-1-(4-fluorophenyl)-5-phthalancarboxamide

t-Butanol (120.0 ml) was added to impure citalopram (thick oil, 80 g) followed by the addition of pulverized potassium hydroxide (27.7 g). The reaction mixture was stirred for about one hour at 75 to 80° C., cooled to 35-40° C. and poured into a brine solution. The product was extracted from the aqueous mixture with dichloromethane and the solvent was evaporated to obtain a residue. Toluene (120 ml) was added to the residue followed by n-hexane (120 ml) under stirring. The suspension was cooled to 5° C. The separated solid was filtered and dried to obtain the title compound of formula II (60 g, purity >98% by HPLC).

### EXAMPLE 2

### Preparation of 1-[3-(dimethylamino)propyl]-1(4-fluorophenyl)-5-phthalancarbonitrilie Hydrobromide

Toluene (320 ml) was added to the above obtained solid (60.0 g) followed by the addition of thionyl chloride (52.2 g). The reaction mixture was stirred at 85 to 95° C. for about one hour and chilled water was added to it. The pH of the mixture was adjusted to 7.5 to 7.8 using aqueous ammonia. The organic layer was separated, washed with water and the solvent was recovered under reduced pressure at 45 to 50° C. Toluene (300 ml) was added to the residue by the addition of 48% aqueous HBr solution (29.5 g) and stirred at 5 to 10° C. After 4 hours of stiring, the upper toluene layer was decanted and fresh toluene (300 ml) was added. The mixture was stirred overnight at 5 to 10° C. The crystalline product obtained was filtered, washed with toluene and dried to obtain citalopram hydrobromide (56.81 g, purity >99.5%, descyano citalopram <0.2% by HPLC).

While the present invention has been described in terms of its specific embodiments, certain modifications and equivalents will be apparent to those skilled in the art and are intended to be included within the scope of the present invention.

We claim:

1. A process for the preparation of pure citalopram hydrobromide of Formula I, 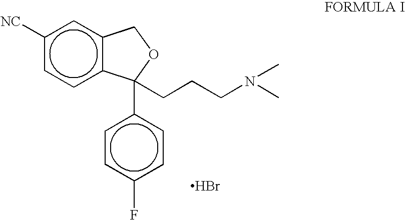
comprising:
i. converting crude citalopram to the corresponding 5-carbamoylphthalane of Formula II, and 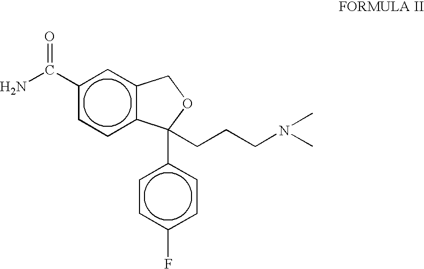
ii. reacting it with a dehydrating agent to obtain citalopram which is converted into its hydrobromide salt.

2. The process of claim 1 wherein the conversion of crude citalopram to 5-carbamoylphthalane of Formula II is achieved by reacting the crude citalopram with a base in the presence of a solvent.

3. The process of claim 2 wherein the base is an alkali metal hydroxide.

4. The process of claim 3 wherein the alkali metal hydroxide is sodium hydroxide, potassium hydroxide, or lithium hydroxide.

5. The process of claim 2 wherein the solvent is alcohol, glycol, glycol ether, or a mixture thereof.

6. The process of claim 5 wherein the solvent is selected from the group consisting of isopropanol, tert-butanol, neo-pentanol, ethylene glycol, diglyme, and mixture(s) thereof.

7. The process of claim 1 wherein the dehydrating is selected from the group consisting of thionyl chloride, phosphoryl chloride, phosphorous pentachloride, polyphosphoric acid, phosphoric acid, and Villsmeier reagent.

8. The process of claim 7 wherein the dehydrating agent is thionyl chloride.

9. The process of claim 1 wherein the dehydration of the compound of formula II is carried out in a solvent.

10. The process of claim 9 wherein the solvent is a hydrocarbon, or a chlorinated hydrocarbon.

11. The process of claim 10 wherein the solvent is selected from the group consisting of toluene, dichloromethane, and mixture(s) thereof.

12. The process for the preparation of citalopram hydrobromide of claim 1 containing less than 0.2% descyano citalopram.

13. Citalopram hydrobromide containing less than 0.2% descyano citalopram.
